(19) United States
(12) Patent Application Publication
(10) Pub. No.: US 2004/0146883 A1
(43) Pub. Date: Jul. 29, 2004

(54) Methods for prenatal diagnosis

(75) Inventor: Giulia Kennedy, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/650,332

(22) Filed: Aug. 27, 2003

Related U.S. Application Data
(60) Provisional application No. 60/443,499, filed on Jan. 28, 2003.

(51) Int. Cl. C12Q 1/68

(52) U.S. Cl. 435/6

(57) ABSTRACT

Methods for prenatal diagnosis are provided. Prenatal DNA sample is genotyped on microarrays and genetic abnormalities are diagnosed.

### REFERENCES TO RELATED APPLICATIONS

This application claims the priority of U.S. Provisional Application Serial No. 60/443,499 filed on Jan. 28, 2003, incorporated herein in its entirety by reference for all purposes.

### FIELD OF THE INVENTION

The present invention relates to genetic analysis. In particular, in one aspect of the invention, prenatal diagnostic methods are provided.

### BACKGROUND OF THE INVENTION

Amniocentesis followed by karyotype analysis is a widely used method for detecting chromosome abnormalities in unborn fetuses. Amniotic fluid is withdrawn from the maternal uterus, and fetal amniocytes are cultured in the lab. Chromosome spreads are then prepared from the cultured cells and visually inspected. While gross chromosomal abnormalities such as large translocations, amplifications and deletions, and extra or missing chromosomes are readily detected, smaller abnormalities are not detected. Furthermore, this diagnosis can take 3-4 weeks while fetal cells are cultured to sufficient numbers in the lab.

### SUMMARY OF THE INVENTION

The use of WGSA (Whole Genome Sampling Assay) genotyping technology, in conjunction with current prenatal diagnosis methods, can be used to identify chromosomal abnormalities at a much higher resolution than is currently available. In one aspect of the invention, a method for prenatal diagnosis is provided, comprising the steps of obtaining a prenatal nucleic acid sample, genotyping at least 500, 1000, 5000, 10000 SNPs in said sample and analyzing the genotypes to determine chromosomal abnormalities. The prenatal nucleic acid sample may be derived from fetal cells obtained by amniocentesis, chorionic villus sampling or by drawing blood from the fetal umbilical cord. The step of genotyping is performed on a solid support, preferably on a microarray. In other embodiments, at least 100 ng, 150 ng, 200 ng, 250 ng of genomic DNA are analyzed.

In another aspect, a kit for prenatal diagnosis is provided, comprising reagents for obtaining a prenatal nucleic acid sample, reagents for genotyping at least 500, 1000, 5000, 10000 SNPs in said sample and reagents for analyzing the genotypes to determine chromosomal abnormalities.

Defects such as nucleic acid amplification, deletion, translocation, extra or missing chromosomal segments, etc. can be diagnosed. The low amount of input DNA required in the WGSA assay would mean that fewer fetal cells would be needed for the analysis, resulting in a faster diagnosis.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 is a schematic showing an exemplary process for prenatal diagnosis.

FIG. 2 shows an exemplary process for performing SNP genotyping.

FIG. 3 is a schematic showing an exemplary process for data analysis.

### DETAILED DESCRIPTION OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the Affymetrix website.

The present invention also contemplates many uses for polymers attached to solid substrates. These uses include gene expression monitoring, profiling, library screening, genotyping and diagnostics. Gene expression monitoring and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Serial No. 60/319,253, Ser. No. 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188, and 5,333,675, and each of which is incorporated herein by reference in their entireties for all purposes. The sample may be amplified on the array. See, for example, U.S. Pat. No. 6,300,070 and U.S. Ser. No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self-sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Ser. Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2^{nd} Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Serial No. 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Serial No. 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2^{nd} ed., 2001). See U.S. Pat. No. 6,420,108.

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Ser. Nos. 10/063,559 (United States Publication No. US20020183936), 60/349,546, 60/376,003, 60/394,574 and 60/403,381.

Definitions

The following terms are intended to have the following general meanings as used herein.

Array: an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array: an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above.

“Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers.

Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98% to 100%. Alternatively, substantial complementarity exists when an RNA or DNA strand will hybridize under selective hybridization conditions to its complement. Typically, selective hybridization will occur when there is at least about 65% complementarity over a stretch of at least 14 to 25 nucleotides, preferably at least about 75%, more preferably at least about 90% complementarity. See. e.g., M. Kanehisa Nucleic Acids Res. 12:203 (1984), incorporated herein by reference.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Effective amount refers to an amount sufficient to induce a desired result.

Genome is all the genetic material in the chromosomes of an organism. DNA derived from the genetic material in the chromosomes of a particular organism is genomic DNA. A genomic library is a collection of clones made from a set of randomly generated overlapping DNA fragments representing the entire genome of an organism.

Hybridization conditions will typically include salt concentrations of less than about 1M, more usually less than about 500 mM and preferably less than about 200 mM. Hybridization temperatures can be as low as 5° C., but are typically greater than 22° C., more typically greater than about 30° C., and preferably in excess of about 37° C. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone.

Hybridizations, e.g., allele-specific probe hybridizations, are generally performed under stringent conditions. For example, conditions where the salt concentration is no more than about 1 Molar (M) and a temperature of at least 25° C., e.g., 750 mM NaCl, 50 mM NaPhosphate, 5 mM EDTA, pH 7.4 (5×SSPE)and a temperature of from about 25° C. to about 30° C.

Hybridizations are usually performed under stringent conditions, for example, at a salt concentration of no more than 1 M and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see, for example, Sambrook, Fritsche and Maniatis. “Molecular Cloning A laboratory Manual” 2^{nd} Ed. Cold Spring Harbor Press (1989) which is hereby incorporated by reference in its entirety for all purposes above.

The term “hybridization” refers to the process in which two single-stranded polynucleotides bind non-covalently to form a stable double-stranded polynucleotide; triple-stranded hybridization is also theoretically possible. The resulting (usually) double-stranded polynucleotide is a “hybrid.” The proportion of the population of polynucleotides that forms stable hybrids is referred to herein as the “degree of hybridization.”

Hybridization probes are oligonucleotides capable of binding in a base-specific manner to a complementary strand of nucleic acid. Such probes include peptide nucleic acids, as described in Nielsen et al., Science 254, 1497-1500 (1991), and other nucleic acid analogs and nucleic acid mimetics. See U.S. Pat. No. 6,156,501.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Preferably, an isolated nucleic acid comprises at least about 50, 70, 80, 90, 95, 99, 99.5% (on a molar basis) of all macromolecular species present. Most preferably, the object species is purified to essential homogeneity (contaminant species cannot be detected in the composition by conventional detection methods).

Ligand: A ligand is a molecule that is recognized by a particular receptor. The agent bound by or reacting with a receptor is called a “ligand,” a term which is definitionally meaningful only in terms of its counterpart receptor. The term “ligand” does not imply any particular molecular size or other structural or compositional feature other than that the substance in question is capable of binding or otherwise interacting with the receptor. Also, a ligand may serve either as the natural ligand to which the receptor binds, or as a functional analogue that may act as an agonist or antagonist. Examples of ligands that can be investigated by this invention include, but are not restricted to, agonists and antagonists for cell membrane receptors, toxins and venoms, viral epitopes, hormones (e.g., opiates, steroids, etc.), hormone receptors, peptides, enzymes, enzyme substrates, substrate analogs, transition state analogs, cofactors, drugs, proteins, and antibodies.

Linkage disequilibrium or allelic association means the preferential association of a particular allele or genetic marker with a specific allele, or genetic marker at a nearby chromosomal location more frequently than expected by chance for any particular allele frequency in the population. For example, if locus X has alleles a and b, which occur equally frequently, and linked locus Y has alleles c and d, which occur equally frequently, one would expect the combination ac to occur with a frequency of 0.25. If ac occurs more frequently, then alleles a and c are in linkage disequilibrium. Linkage disequilibrium may result from natural selection of certain combination of alleles or because an allele has been introduced into a population too recently to have reached equilibrium with linked alleles.

Mixed population or complex population: refers to any sample containing both desired and undesired nucleic acids. As a non-limiting example, a complex population of nucleic acids may be total genomic DNA, total genomic RNA or a combination thereof. Moreover, a complex population of nucleic acids may have been enriched for a given population but include other undesirable populations. For example, a complex population of nucleic acids may be a sample which has been enriched for desired messenger RNA (mRNA) sequences but still includes some undesired ribosomal RNA sequences (rRNA).

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

mRNA or mRNA transcripts: as used herein, include, but are not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, an RNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

Nucleic acid library or array is an intentionally created collection of nucleic acids which can be prepared either synthetically or biosynthetically and screened for biological activity in a variety of different formats (e.g., libraries of soluble molecules; and libraries of oligos tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleoside sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

Nucleic acids according to the present invention may include any polymer or oligomer of pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA) which may be isolated from natural sources, recombinantly produced or artificially synthesized and mimetics thereof. A further example of a polynucleotide of the present invention may be peptide nucleic acid (PNA). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

Probe: A probe is a surface-immobilized molecule that can be recognized by a particular target. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Primer is a single-stranded oligonucleotide capable of acting as a point of initiation for template-directed DNA synthesis under suitable conditions e.g., buffer and temperature, in the presence of four different nucleoside triphosphates and an agent for polymerization, such as, for example, DNA or RNA polymerase or reverse transcriptase. The length of the primer, in any given case, depends on, for example, the intended use of the primer, and generally ranges from 15 to 30 nucleotides. Short primer molecules generally require cooler temperatures to form sufficiently stable hybrid complexes with the template. A primer need not reflect the exact sequence of the template but must be sufficiently complementary to hybridize with such template. The primer site is the area of the template to which a primer hybridizes. The primer pair is a set of primers including a 5′ upstream primer that hybridizes with the 5′ end of the sequence to be amplified and a 3′ downstream primer that hybridizes with the complement of the 3′ end of the sequence to be amplified.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Receptor: A molecule that has an affinity for a given ligand. Receptors may be naturallyoccurring or manmade molecules. Also, they can be employed in their unaltered state or as aggregates with other species. Receptors may be attached, covalently or noncovalently, to a binding member, either directly or via a specific binding substance. Examples of receptors which can be employed by this invention include, but are not restricted to, antibodies, cell membrane receptors, monoclonal antibodies and antisera reactive with specific antigenic determinants (such as on viruses, cells or other materials), drugs, polynucleotides, nucleic acids, peptides, cofactors, lectins, sugars, polysaccharides, cells, cellular membranes, and organelles. Receptors are sometimes referred to in the art as anti-ligands. As the term receptors is used herein, no difference in meaning is intended. A “Ligand Receptor Pair” is formed when two macromolecules have combined through molecular recognition to form a complex. Other examples of receptors which can be investigated by this invention include but are not restricted to those molecules shown in U.S. Pat. No. 5,143,854, which is hereby incorporated by reference in its entirety.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations. See U.S. Pat. No. 5,744,305 for exemplary substrates.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term target is used herein, no difference in meaning is intended. A “Probe Target Pair” is formed when two macromolecules have combined through molecular recognition to form a complex.

Prenatal: Occurring, existing or performed before birth. The term ‘antenatal’ may be used interchangeably with ‘prenatal’.

Prenatal Diagnosis: Determination of a pathological or a physiological state such as a disease in the embryo, fetus, or pregnant female before birth.

Amniocentesis: Process of sampling the fluid in the amniotic sac.

Karyotyping: Process of producing a karyotype for a cell or a cell line. For example, for human cells, a karyotype shows whether the normal pattern of 46 chromosomes is present or not.

Congenital: Existing at and usually before, birth.

Down's Syndrome: Formerly known as Mongolism, Down's syndrome is a congenital disorder in which a person is born with three copies of chromosome 21 (trisomy 21). Clinical features include moderate to severe mental retardation, slanting eyes, a broad short skull, broad hands and short fingers. Other congenital abnormalities include heart defects, oesophageal atresia and an increased incidence of acute lymphocytic leukaemia. Amniocentesis can be used to detect trisomy 21 in the first few months of pregnancy. Factors that may predispose a child to Down's syndrome include a prior child with Down's syndrome and mothers who become pregnant after the age of 40. Incidence of this disorder is about 1 in 1000 births.

Spina bifida: A congenital cleft in the spinal column, characterized by the absence of the vertebral arches through which the spinal membranes and spinal cord may protrude.

Chorionic villus sampling (CVS): A procedure used to diagnose certain birth defects in the first trimester of pregnancy after an ultrasound examination. The procedure involves inserting a small catheter (tube) through the cervix and into the developing placenta and gently suctioning a small amount of placental tissue into a syringe. Both the placental and fetal tissues originate from the same cell line and are genetically identical. Thus, by obtaining a tiny sample of the chorionic villi from the placenta, one can determine certain genetic characteristics of the fetus.

WGSA (Whole Genome Sampling Assay) Genotyping Technology: A technology that allows the genotyping of thousands of SNPs simultaneously in complex DNA without the use of locus-specific primers. In this technique, genomic DNA, for example, is digested with a restriction enzyme of interest and adaptors are ligated to the digested fragments. A single primer corresponding to the adaptor sequence is used to amplify fragments of a desired size, for example, 500-2000 bp. The processed target is then hybridized to nucleic acid arrays comprising SNP-containing fragments/probes. WGSA is disclosed in, for example, U.S. Provisional Application Serial Nos. 60/319,685, 60/453,930, 60/454,090 and 60/456,206, 60/470,475, U.S. patent application Ser. Nos. 09/766,212, 10/316,517, 10/316,629, 10/463,991, 10/321,741, 10/442,021 and 10/264,945, each of which is hereby incorporated by reference in its entirety for all purposes.

II. Prenatal Diagnosis Using Whole Genome Sampling Assay

Prenatal or antenatal diagnosis or testing is commonly used to diagnose abnormalities in the fetus, such as the presence of chromosomal translocations, deletions and/or amplifications, or an extra, missing or rearranged chromosome. About 85-90 percent of babies born with major abnormalities have either trisomy 21 (Down's syndrome), trisomy 18, trisomy 13, or an abnormal number of sex chromosomes.

Fetal cells for analysis can be obtained by amniocentesis, chorionic villus sampling (CVS), or drawing blood from the fetal umbilical cord. Amniocentesis is the most commonly used method to collect fetal cells. The procedure is usually performed in the 15th week of pregnancy or later, but can sometimes be performed as early as the 11th week. A needle is inserted through the mother's abdominal wall and fetal cells (amniocytes) are removed from the amniotic sac (the fluid-filled sack surrounding the fetus). Karyotyping or other routine cytogenetic testing method(s) can be performed to assess all cells for chromosome abnormalities. This procedure, which usually takes about 7-10 days, has been the gold standard in the field of prenatal testing. However, the need for a procedure that provides rapid and accurate results is essential in cases when an abnormality is suspected late in a pregnancy or when complications require an immediate diagnosis.

In standard cytogenetic testing, the fetal cells obtained by amniocentesis are cultured in a laboratory incubator under special conditions so that the number and structure of all the chromosomes in the cells can be studied under a microscope. Images of the chromosomes are made and the chromosomes are paired according to structure and size, creating a karyotype. The time-consuming step in this procedure is the time required by cells to grow enough to ensure that each and every chromosome can be studied.

In karyotyping, one can count the number of chromosomes or look for structural changes in chromosomes, or do both. This provides an indication of genetic changes associated with increased risk for disease. The test can be performed on a sample of blood, bone marrow, amniotic fluid, or placental tissue. Abnormalities can be identified through the number or arrangement of the chromosomes.

Conventional prenatal diagnostic methods, such as those described above, usually take a few days to a few weeks to carry out. In many cases, only a limited number of cells are available for DNA isolation and diagnosis. Moreover, the period for culturing amniocytes or chorionic villi (for example) is usually around 10-14 days. Such a long time frame could sometimes be unacceptable for a clinician or a patient. In addition, while the above techniques allow detection of large chromosomal abnormalities, smaller aberrations such as deletions, amplifications, translocations and rearrangements may be missed.

In one aspect of the invention, methods for prenatal genetic diagnosis are provided. The methods can be used to identify chromosomal abnormalities at a much higher resolution than is currently available, are easier to automate and less prone to error. The low amount of input DNA required in the WGSA assay (about 250 ng of genomic DNA) would mean that fewer fetal cells would be needed for the analysis, resulting in a faster diagnosis.

The practice of the present invention may employ, unless otherwise indicated, conventional techniques and descriptions of clinical diagnosis, organic chemistry, polymer technology, molecular biology (including recombinant techniques), cell biology, biochemistry, and immunology, which are within the skill of the art. Such conventional techniques include amniocentesis, chorionic villus sampling, fluorescence in situ hybridization, polymer array synthesis, hybridization, ligation, and detection of hybridization using a label. Specific illustrations of suitable techniques can be had by reference to the example herein below. However, other equivalent conventional procedures can also be used. Such conventional techniques and descriptions can be found in e.g. Diagnosis of Fetal Abnormalities: The 18-23-Week Scan by Kypros H. Nicolaides and Gianluigi Pilu (Eds), ISBN: 1850704929; The 11-14-Week Scan: The Diagnosis of Fetal Abnormalities by Kypros H. Nicolaides, Neil J. Sebire, Rosalinde J. M. Snijders, K. H. Nicolaides, N. Sebire, R. J. M. Snijders, ISBN: 185070743X; Antenatal and Neonatal Screening, by Nicholas J. Wald and Ian Leck (Eds), ISBN: 0192628267; and standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W.H. Freeman Pub., New York, N.Y., all of which are herein incorporated in their entirety by reference for all purposes.

The present invention also contemplates sample preparation and genotyping methods in certain preferred embodiments. Traditional sources of high-quality DNA for prenatal diagnosis have been chorionic villi samples, fetal blood, or amniotic fluid. Adequate amounts of DNA can be extracted from amniotic fluid cells beginning at 8 weeks gestation, and these samples are suitable for prenatal diagnosis using PCR. Specific illustrations of suitable DNA isolation and purification techniques can be found in, for example, Lewandowska-Skarbek et al. “Rapid Isolation of PCR-ready DNA for Prenatal Diagnosis Using the MasterAmp™ Buccal Swab DNA Extraction Solution” Epicentre Technologies (available online at Epicentre's website); Wei et al. “Screening cell-free fetal DNA in maternal plasma” Qiagen News, Issue No. 4, 2001 (available online at Qiagen's website); Bianchi et al. “Large Amounts of Cell-free Fetal DNA are present in amniotic fluid.” Clinical Chemistry 47, No. 10 (2001) 1867-1869, each of which is hereby incorporated by reference in its entirety for all purposes.

The GeneChip® Mapping 10K Array from Affymetrix Inc. (Santa Clara, Calif.) provides a suitable substrate for hybridization studies. The 10K array provides a robust assay for genotyping more than 10,000 SNPs using a single PCR primer. The amount of input DNA required for genotyping is only about 250 ng. The nature of the assay eliminates the need for locus-specific PCR. The assay includes automated genotype calling with an accuracy of >99.6% and reproducibility of 99.99%. The 10K array contains approximately 11,555 SNPs, with an average distance of 210 kb between markers. The 10,000 SNPs genotyping assay includes many advantages over the most commonly used genotyping techniques. Technical information pertaining to the 10K array and genotyping assay can be obtained from the 10K Manual and Data Sheet, available on the website of Affymetrix Inc, incorporated herein by reference for all purposes.

However, one of skill in the art would appreciate that the scope of the invention is not limited to any specific SNP genotyping methods. In contrast, all range of suitable methods are contemplated.

The present invention contemplates rapid diagnosis of inherited diseases. These include diseases diagnosed by restriction-site variation, such as Duchenne's muscular dystrophy and sickle cell anemia, those due to a collection of known mutations, such as beta-thalassemia, and those due to gene deletion, such as alpha-thalassemia.

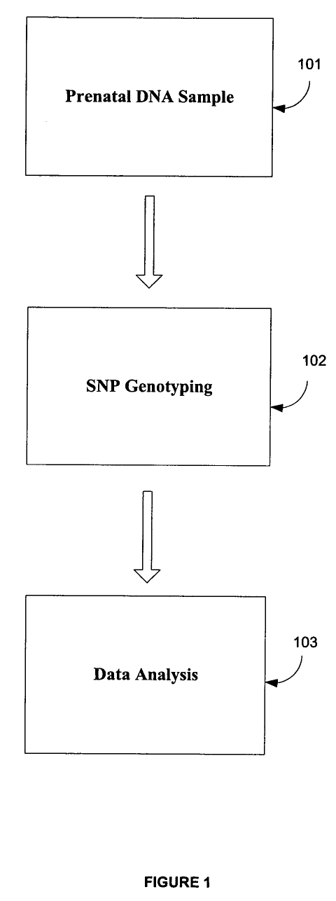

FIG. 1 provides a broad overview of the prenatal diagnosis methods in one aspect of the present invention. Prenatal DNA sample is isolated from cells and/or tissues that may be obtained by various methods including amniocentesis, chorionic villus sampling (CVS) or drawing blood from the fetal umbilical cord (101). Numerous suitable methods are availble for the purification of DNA. In some cases, about 250 ng of input DNA is sufficient for SNP genotyping. The DNA or samples derived from the DNA is analyzed for at least 100, 500, 1000, 5000, 10,000 SNPs (102). The SNP genotypes are analyzed to determine the presence of any genetic abnormalities (103).

In preferred embodiments, the genotyping is carried out using oligonucleotide probes. The probes are typically immobilized in a microarray format on a substrate, on optical fibers or on beads. Affymetrix GeneChip® 10K mapping array provides a commercially available example for such probes. Exemplary probe design/tiling strategy is described in, e.g., U.S. Provisional Application Serial Nos. 60/319,685, 60/453,930, 60/454,090 and 60/456,206, 60/470,475, U.S. patent application Ser. Nos. 09/766,212, 10/316,517, 10/316,629, 10/463,991, 10/321,741, 10/442,021 and 10/264,945, each of which is hereby incorporated by reference in its entirety for all purposes.

In some embodiments, probe intensities are used directly to detect genetic abnormalities.

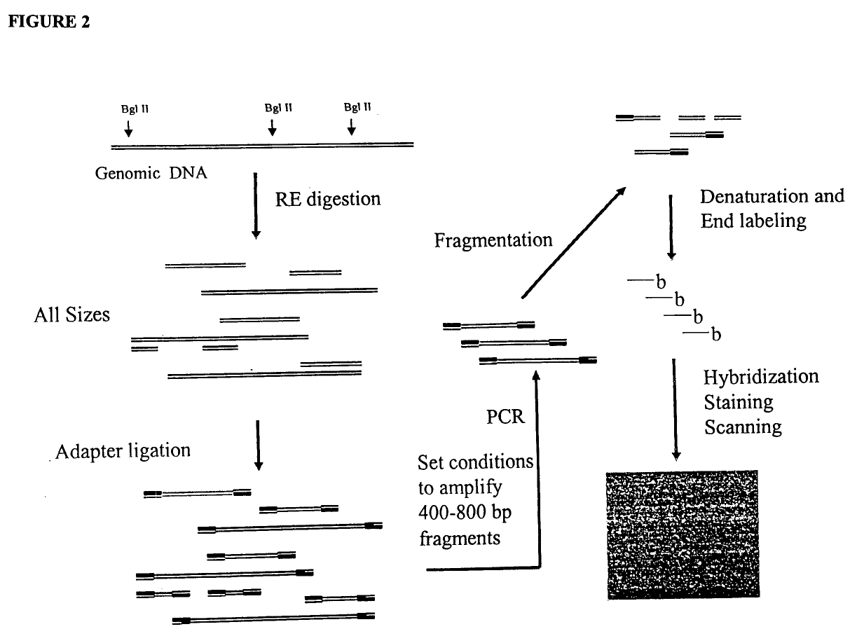

In some embodiments, the SNP genotyping is carried out using the Whole Genome Sampling Assay (WGSA). FIG. 2 provides an overview of the WGSA. For example, about 250 ng of total genomic DNA (prenatal DNA sample and normal adult genomic DNA) is digested with a restriction enzyme (such as XbaI) and ligated to adapters that recognize the cohesive four base pair (bp) overhangs. All fragments resulting from restriction enzyme digestion, regardless of size, are substrates for adapter ligation. A generic primer that recognizes the adapter sequence is used to amplify adapter ligated DNA fragments. Optimized PCR conditions preferentially amplify fragments in the 250 to 1000 bp size range. The amplified DNA is then fragmented, labeled, and hybridized to the GeneChip Mapping 10K Array. WGSA is disclosed in, for example, U.S. Provisional Application Serial Nos. 60/319,685, 60/453,930, 60/454,090 and 60/456,206, 60/470,475, U.S. patent application Ser. Nos. 09/766,212, 10/316,517,10/316,629, 10/463,991, 10/321,741, 10/442,021 and 10/264,945, each of which is hereby incorporated by reference in its entirety for all purposes.

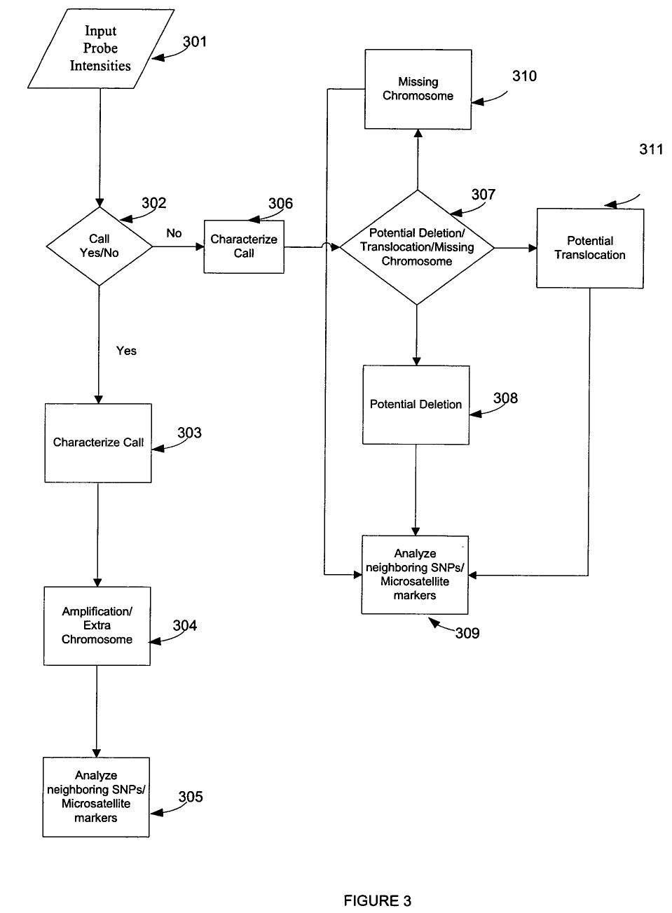

One aspect of the data analysis process of the invention is provided in FIG. 3. Probe intensities are inputted and the presence (Yes) or absence (No) of a genpotype/SNP is determined (called). Data obtained from genotyping prenatal DNA sample may be compared to data obtained from genotyping normal adult genomic DNA, which serves as the reference. In some instances, intensities may be used directly to determine genetic abnormalities. A positive call could mean amplification or the presence of an extra chromosome. Perfect match (PM) intensity is used to detect amplifications. Neighboring SNPs or microsatellite markers (other markers) may be analyzed. If there are neighboring SNPs or other markers found contiguously with the SNP in question, that are not present in the reference sample, it confirms amplification of the SNP. It would be evident to one of skill in the art that the robustness of this test is directly proportional to the number of SNPs found contiguously amplified, i.e. the more the number of SNPs that are contiguously amplified, the more robust the conclusion of genetic amplification is.

A negative call could mean potential deletion or translocation or a missing chromosome/chromosomal segment. In one embodiment, Discrimination Ratio or DR [(PM−MM)/(PM+MM)] may be used to detect deletions. As in the case of a genetic amplification, information from neighboring SNPs/microsatellite markers/other markers is obtained. The power of the test is directly proportional to the number of SNPs found contiguously deleted.

In another aspect of the invention, computer systems and software products are provided for prenatal diagnosis. The computer systems are typically digital computers with at least one central processing unit coupled with a memory. The computers are suitable for executing computer codes that performs the method of the invention. The computer software products of this invention typically contain a computer readable medium (e.g., CD/DVD ROM, floppy disk, optical magnetic storage device, etc.). Computer codes that perform the methods of the invention are stored in the computer readable medium.

In one exemplary computerized data analysis method (embodied in computer systems and software products), probe intensities are inputted (301). The intensities are analyzed to make a genotyping call (302). A positive call (303), suggests for example, amplification or the presence of an extra chromosome (304). Neighboring SNPs and/or microsatellite markers, if available are characterized in order to confirm the exact genotype (305). A negative call (306) suggests, for example, a potential deletion (308) or a missing chromosome (310) or a potential translocation (311). Again, neighboring SNPs and/or microsatellite markers, if available are characterized in order to confirm the exact genotype (309).

It is to be understood that the above description is intended to be illustrative and not restrictive. Many variations of the invention will be apparent to those of skill in the art upon reviewing the above description. All cited references, including patent and non-patent literature, are incorporated herein by reference in their entireties for all purposes.

What is claimed is:

1. A method for prenatal diagnosis comprising:
obtaining a prenatal nucleic acid sample
genotyping at least 5000 SNPs in said sample
analyzing the genotypes to determine chromosomal abnormalities.

2. The method of claim 1 wherein the prenatal nucleic acid sample is derived from fetal cells obtained by amniocentesis.

3. The method of claim 1 wherein the prenatal nucleic acid sample is derived from fetal cells obtained by chorionic villus sampling.

4. The method of claim 1 wherein the prenatal nucleic acid sample is derived from fetal cells obtained by drawing blood from the fetal umbilical cord.

5. The method of claim 1 wherein the step of genotyping is performed on a solid support.

6. The method of claim 5 wherein the step of genotyping is performed on a microarray.

7. The method of claim 1 wherein at least 250 ng of genomic DNA is analyzed.

8. The method of claim 7 wherein at least 200 ng of genomic DNA is analyzed.

9. The method of claim 8 wherein at least 150 ng of genomic DNA is analyzed.

10. The method of claim 9 wherein at least 100 ng of genomic DNA is analyzed.

11. The method of claim 1 wherein the prenatal nucleic acid sample is amplified prior to genotyping.

12. A kit for prenatal diagnosis comprising
reagents for obtaining a prenatal nucleic acid sample
reagents for genotyping at least 5000 SNPs in said sample
reagents for analyzing the genotypes to determine chromosomal abnormalities.
